(19) United States
(12) Patent Application Publication
(10) Pub. No.: US 2025/0268677 A1
(43) Pub. Date: Aug. 28, 2025

(54) SYSTEMS AND METHODS FOR POSITIONING MEDICAL INSTRUMENTS

(71) Applicant: Intuitive Surgical Operations, Inc., Sunnyvale, CA (US)

(72) Inventor: David W. Bailey, Sunnyvale, CA (US)

(73) Assignee: Intuitive Surgical Operations, Inc., Sunnyvale, CA (US)

(21) Appl. No.: 18/858,082

(22) PCT Filed: Apr. 20, 2023
(86) PCT No.: PCT/US2023/019230

§ 371 Date: Oct. 18, 2024

Related U.S. Application Data
(60) Provisional application No. 63/332,918, filed on Apr. 20, 2022.

(51) Int. Cl. A61B 34/00; A61B 18/14; A61B 34/20; A61B 34/30

(52) U.S. Cl. CPC A61B 34/71; A61B 18/1492; A61B 34/20; A61B 34/30; A61B 34/76; A61B 2034/2055; A61B 2034/301; A61B 2034/305

(57) ABSTRACT

A backend mechanism of a control system for an elongated medical instrument includes a tool holder to retain and move a tool relative to the elongated medical instrument.

### CROSS-REFERENCE TO RELATED APPLICATIONS

This application claims the benefit of priority under 35 U S.C. § 119 (e) to U.S. Provisional Patent Application Ser. No. 63/332,918, filed on Apr. 20, 2022, which is hereby incorporated by reference herein in its entirety.

### FIELD

The present disclosure is directed to systems and methods for controlling medical instruments.

### BACKGROUND

Minimally invasive medical techniques are intended to reduce the amount of tissue that is damaged during medical procedures, thereby reducing patient recovery time, discomfort, and harmful side effects. Such minimally invasive techniques may be performed through natural orifices in a patient anatomy or through one or more surgical incisions. Through these natural orifices or incisions physician may insert minimally invasive medical instruments (including imaging surgical, diagnostic, therapeutic, or biopsy instruments) to reach a target tissue location. One such minimally invasive technique is to use a flexible and/or steerable elongate device, such as a flexible catheter, that can be inserted into anatomic passageways and navigated toward a region of interest within the patient anatomy. Control of such an elongate device by medical personnel involves the management of several degrees of freedom including at least the management of insertion and retraction of the elongate device as well as steering of the device.

### SUMMARY

Consistent with some embodiments, a medical instrument may include a housing and an elongate member axially fixed to and extending distally from the housing. The elongate member may include a channel extending through the elongate member. The medical instrument may also include a tool holder moveably mounted to the housing and configured to accept and retain a tool inserted through the tool holder and into the channel of the elongate member. Movement of the tool holder may move the tool relative to the elongate member.

Consistent with some embodiments, a method for controlling movement of a medical instrument may include positioning and retaining a tool in a tool holder moveably mounted to a housing. The tool may extend into a channel extending through an elongate member of a medical instrument. The method may further include moving the tool holder relative to the housing while maintaining an axial position of the elongate member relative to the housing to move the tool relative to the elongate member.

Consistent with some embodiments, a medical instrument may include a housing and an elongate member axially fixed to and extending distally from the housing, the elongate member including a channel extending through the elongate member. The medical instrument may also include a tool holder moveably mounted to the housing and configured to accept and retain a tool inserted through the tool holder and into the channel of the elongate member. The tool holder is configured to rotate relative to the housing and the elongate member and rotation of the tool holder rotates the tool relative to the elongate member.

It should be appreciated that the foregoing concepts, and additional concepts discussed below, may be arranged in any suitable combination, as the present disclosure is not limited in this respect. Further, other advantages and novel features of the present disclosure will become apparent from the following detailed description of various non-limiting embodiments when considered in conjunction with the accompanying figures.

In cases where the present specification and a document incorporated by reference include conflicting and/or inconsistent disclosure, the present specification shall control. If two or more documents incorporated by reference include conflicting and/or inconsistent disclosure with respect to each other, then the document having the later effective date shall control.

### BRIEF DESCRIPTION OF DRAWINGS

The accompanying drawings are not intended to be drawn to scale. In the drawings, each identical or nearly identical component that is illustrated in various figures may be represented by a like numeral. For purposes of clarity, not every component may be labeled in every drawing. In the drawings:

FIG. 1 is a simplified diagram of a teleoperated medical system adjacent a patient according to some embodiments.

FIG. 2A is a simplified diagram of a medical instrument system according to some embodiments.

FIG. 2B is a simplified diagram of a medical instrument with an extended medical tool according to some embodiments.

FIGS. 3A and 3B are simplified diagrams of side views of a medical instrument mounted on an insertion assembly and positioned to treat a patient according to some embodiments.

FIG. 4 is diagram of a backend mechanism of a medical instrument according to some embodiments.

FIG. 5 is diagram of a backend mechanism of a medical instrument in a partially exploded configuration according to some embodiments.

FIG. 6 is diagram of a backend mechanism of a medical instrument in a partially exploded configuration according to some embodiments.

FIG. 7 is a diagram of a portion of a backend mechanism of a medical instrument according to some embodiments.

FIG. 8 is a diagram of a portion of a backend mechanism of a medical instrument according to some embodiments.

FIG. 9 is a diagram of a portion of a backend mechanism of a medical instrument according to some embodiments.

FIG. 10 is a flow chart showing an example method of moving an elongate member and medical tool of a backend mechanism according to some implementations.

Embodiments of the present disclosure and their advantages are best understood by referring to the detailed description that follows. It should be appreciated that like reference numerals are used to identify like elements illustrated in one or more of the figures, wherein showings therein are for purposes of illustrating embodiments of the present disclosure and not for purposes of limiting the same.

### DETAILED DESCRIPTION

During minimally invasive medical techniques, an elongate member (e.g., catheter) may be navigated to a target site. A medical tool may then be inserted into a lumen of the elongate member and navigated to a distal end of the elongate member and positioned proximate to the target site. Once the medical tool has reached the distal end of the elongate member, the elongate member is retracted while the medical tool is extended to expose the tip of the medical tool and maintain the position of the medical tool proximate to the target tissue site.

In an ablation procedure, for example, an ablation tool may be navigated through the elongate member to the distal end of the elongate member, or past the distal end of the elongate member, such that the ablation tool is placed proximate to the target tissue while still within the lumen of the elongate member. The elongate member may then be retracted while the ablation tool is simultaneously extended to expose the distal end of the ablation tool and maintain the position of the ablation tool proximate to the target tissue. Once exposed from the distal end of the elongate member, the ablation tool may perform an ablation procedure on the target tissue. Retraction of the elongate member during the ablation procedure exposes the energy producing portion of the ablation tool to the tissue and prevents the ablation tool from damaging the elongate member.

The simultaneous retraction of the elongate member and extension of the medical tool may be performed by an operator manually controlling the position of both the elongate member and the medical tool. However, such coordination requires awkward and difficult control using two hands to perform the two different operations to maintain the tool in a desired location during retraction of the elongate member. This task is further complicated by the fact that, while retracting the instrument, the user also needs to steer the catheter to maintain the tool tip position. This effectively makes the task a “three handed” operation.

In view of the above, systems and methods facilitate the coordination of exposing a distal end of a medical tool disposed within an elongate member while maintaining the position of the medical tool relative to the target tissue site. In some embodiments, a medical system may automate the workflow of positioning the elongate member and the medical tool. For example, a user (e.g., physician or other medical care provider) may use a medical system to navigate an elongate member and a medical tool to a desired position in a patient. When in the desired position, the user may direct the system to automate and control the opposing movements of the elongate member and the medical tool to expose the distal end portion of the medical tool while maintaining the medical tool in the desired position and/or steering the elongate member. In a non-limiting example, a user may press a button on the system to change the system from a “drive mode” (e.g., to direct one or more drive components to control the insertion of the elongate member to navigate the elongate member to a target site) to an “expose mode” (e.g., to direct one or more drive components to control the position and exposure of the medical tool by simultaneously advancing the medical tool while retracting the elongate member such that the medical tool remains substantially stationary in a desired position).

In some embodiments, a medical system may control a medical tool position relative to an elongate member through which the medical tool extends. In some embodiments, a backend mechanism of a medical system may include a proximal portion of an elongate member that is axially fixed to a housing of the backend mechanism. As such, the elongate member may be moved in an axial direction, to advance or retract the elongate member, by moving the backend mechanism in the desired direction. In some embodiments, the backend mechanism may also include a tool holder that is moveably mounted to the housing and configured to accept and retain a medical tool (e.g., an ablation tool). The tool holder may have an opening at a proximal end providing access to a channel that extends at least partially through the tool holder and connected to a channel that extends through the elongate member. As such, the medical tool may be inserted through the tool holder and into the channel of the elongate member. Because the tool holder retains the medical tool, movement of the tool holder (e.g., translation, rotation) causes movement of the medical tool relative to the elongate member. For example, in some embodiments, the backend mechanism may be moved in a first direction to cause the elongate member to move in the first direction while the tool holder simultaneously moves in a second direction, opposite the first direction, to cause the medical tool to move in the second direction relative to the elongate member. Such simultaneous movements may be coordinated by the system to keep the medical tool in a fixed position within a patient. For example, in some embodiments, the corresponding movements of the backend mechanism and the tool holder may be equal in magnitude and oriented opposite to each other to maintain the tool substantially stationary. However, motions of these components with non-equal magnitudes and non-stationary operation of the tool may also be used in other embodiments. In either case, the medical system may permit easy positional control of the medical tool relative to the elongate member without any need for manual manipulation of the medical tool.

In some embodiments, the medical system may include various sensors to collect positional data and movement of the backend mechanism, elongate member, and/or medical tool. In some embodiments, the medical system may include a first actuator configured to control movement of a backend mechanism and a second actuator configured to control movement of a tool holder. Based on data input from sensors and a desired retraction of the elongate member, the first and second actuators may be controlled to retract the elongate member in a proximal direction while a corresponding distal movement of the tool is applied to maintain a position of the medical tool.

The medical system may be used for medical procedures such as, but not limited to, surgery, biopsy, ablation, illumination, irrigation, suction, imaging, or any other appropriate medical procedure. In some embodiments, medical tools used with the medical system may include biopsy, ablation, imaging devices, and/or any other appropriate tool. The medical tools may be used in endoscopes, catheters, or any system with an articulable elongated member through which a medical tool may be navigated. It should be noted that any use of a catheter in some embodiments is simply for clarity, and other types of elongate members may be used. In some embodiments, a medical tool may be retained and locked into position by the tool holder using various locking techniques. Such locking techniques may include, but are not limited to compression fittings, detents, clamps, or any other appropriate holder capable of engaging with a medical tool.

In some embodiments, the backend mechanism may include various structures to cause movement of the tool holder, including a rocker arm, rack and pinion, or one or more pulleys. The tool holder may also be configured to move in various directions, including but not limited to: translation including translation in a direction aligned with a longitudinal axis of an associated portion of the elongated member; rotation relative to the longitudinal axis of the elongated member; combinations of the forgoing; and/or any other appropriate type of motion. Sensors configured to track and control movement of the elongate member and the medical tool may include encoders, linear displacement sensors (e.g., Linear Variable Differential Transformer (LVDT)) and/or any other appropriate type of sensor.

For the purposes of promoting an understanding of the principles of the present disclosure, reference will now be made to the embodiments illustrated in the drawings, and specific language will be used to describe the same. In the following detailed description, numerous specific details are set forth to provide a thorough understanding of the disclosed embodiments. However, the embodiments of this disclosure may be practiced without these specific details. In other instances, well known methods, procedures, components, and circuits have not been described in detail so as not to unnecessarily obscure aspects of the embodiments described herein.

Any alterations and further modifications to the described devices, instruments, methods, and any further application of the principles of the present disclosure are fully contemplated as would normally occur to one skilled in the art to which the disclosure relates. For example, the features, components, and/or steps described with respect to one embodiment may be combined with the features, components, and/or steps described with respect to other embodiments of the present disclosure. In addition, dimensions provided herein are for specific examples and it is contemplated that different sizes, dimensions, and/or ratios may be utilized to implement the concepts of the present disclosure. To avoid needless descriptive repetition, one or more components or actions described in accordance with one illustrative embodiment can be used or omitted as applicable from other illustrative embodiments. For the sake of brevity, the numerous iterations of these combinations will not be described separately. For simplicity, in some instances the same reference numbers are used throughout the drawings to refer to the same or like parts.

This disclosure describes various instruments and portions of instruments in terms of their state in three-dimensional space. As used herein, the term “position” refers to the location of an object or a portion of an object in a three-dimensional space (e.g., three degrees of translational freedom along Cartesian x-, y-, and z-coordinates). As used herein, the term “orientation” refers to the rotational placement of an object or a portion of an object (three degrees of rotational freedom—e.g., roll, pitch, and yaw). As used herein, the term “pose” refers to the position of an object or a portion of an object in at least one degree of translational freedom and to the orientation of that object or portion of the object in at least one degree of rotational freedom (up to six total degrees of freedom). As used herein, the term “shape” refers to a set of poses, positions, or orientations measured along an object.

Turning to the figures, specific non-limiting embodiments are described in further detail. The various systems, components, features, and methods described relative to these embodiments may be used either individually and/or in any desired combination as the disclosure is not limited to only the specific embodiments described herein.

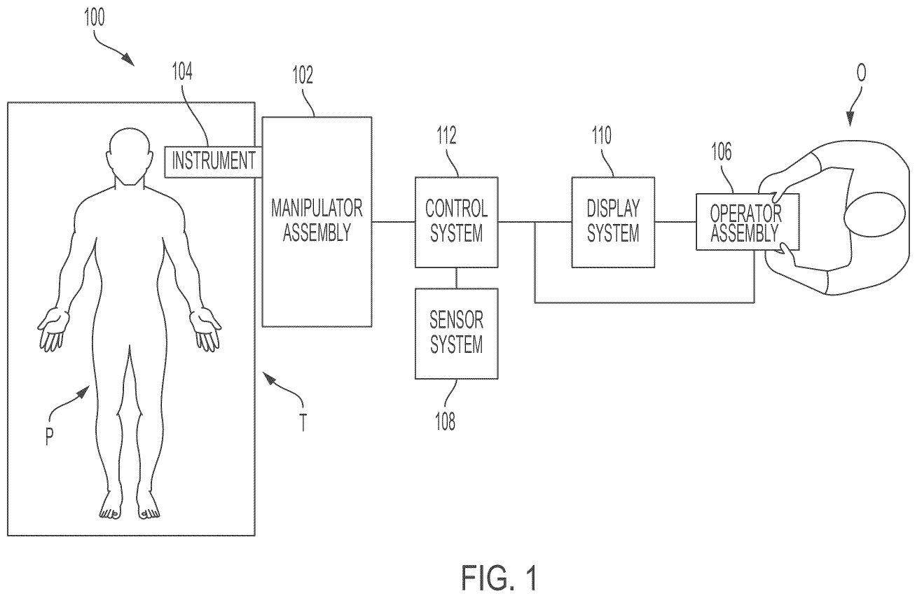

FIG. 1 is a simplified diagram of a teleoperated medical system 100 according to some embodiments. In some embodiments, teleoperated medical system 100 may be suitable for use in, for example, surgical, diagnostic, therapeutic, ablation, or biopsy procedures. While some embodiments are provided herein with respect to such procedures, any reference to medical or surgical instruments and medical or surgical methods is non-limiting. The systems, instruments, and methods described herein may be used for animals, human cadavers, animal cadavers, portions of human or animal anatomy, non-surgical diagnosis, as well as for industrial systems and general robotic or teleoperational systems.

As shown in FIG. 1, medical system 100 generally includes a manipulator assembly 102 for operating a medical instrument 104 in performing various procedures on a patient P. Manipulator assembly 102 is mounted to or near an operating table T. An operator assembly 106 allows an operator O (e.g., a surgeon, a clinician, or a physician as illustrated in FIG. 1) to view the interventional (e.g., surgical) site and to control manipulator assembly 102.

Operator assembly 106 may be located at an operator console which is usually located in the same room as operating table T, such as at the side of a surgical table on which patient P is located. However, operator O can be located in a different room or a completely different building from patient P. Operator assembly 106 generally includes one or more control devices for controlling manipulator assembly 102. The control devices may include any number of a variety of input devices, such as joysticks, trackballs, data gloves, trigger-guns, hand-operated controllers, voice recognition devices, body motion or presence sensors, and/or the like. To provide operator O a strong sense of directly controlling instruments 104 the control devices may be provided with the same degrees of freedom as the associated medical instrument 104. In this manner, the control devices provide physician O with telepresence or the perception that the control devices are integral with medical instruments 104.

In some embodiments, the control devices may have more or fewer degrees of freedom than the associated medical instrument 104 and still provide operator O with telepresence. In some embodiments, the control devices may be manual input devices which move with six degrees of freedom, and which may also include an actuatable handle for actuating instruments (for example, for closing grasping jaws, applying an electrical potential to an electrode, delivering a medicinal treatment, and/or the like).

Manipulator assembly 102 supports medical instrument 104 and may include a kinematic structure of one or more non-servo controlled links (e.g., one or more links that may be manually positioned and locked in place, generally referred to as a set-up structure), and/or one or more servo controlled links (e.g., one more links that may be controlled in response to commands from the control system), and a teleoperational manipulator. Manipulator assembly 102 may include a plurality of actuators or motors that drive inputs on medical instrument 104 in response to commands from the control system (e.g., a control system 112). The actuators may include drive systems that when coupled to medical instrument 104 may advance medical instrument 104 into a naturally or surgically created anatomic orifice. Other drive systems may move the distal end of medical instrument 104 in multiple degrees of freedom, which may include three degrees of linear motion (e.g., linear motion along the X, Y, Z Cartesian axes) and in three degrees of rotational motion (e.g., rotation about the X, Y, Z Cartesian axes). Additionally, the actuators can be used to actuate an articulable end effector of medical instrument 104, such as for grasping tissue in the jaws of a biopsy device and/or the like. Actuator position sensors such as resolvers, encoders, potentiometers, and other mechanisms may provide sensor data to medical system 100 describing the rotation and orientation of the motor shafts. This position sensor data may be used to determine motion of the objects manipulated by the actuators.

Teleoperated medical system 100 may include a sensor system 108 with one or more sub-systems for receiving information about the instruments of manipulator assembly 102. Such sub-systems may include a position/location sensor system (e.g., an electromagnetic (EM) sensor system); a shape sensor system for determining the position, orientation, speed, velocity, pose, and/or shape of a distal end and/or of one or more segments along a flexible body that may make up medical instrument 104; and/or a visualization system for capturing images from the distal end of medical instrument 104.

Teleoperated medical system 100 also includes a display system 110 for displaying an image or representation of the interventional site and medical instrument 104 generated by sub-systems of sensor system 108. Display system 110 and operator assembly 106 may be oriented so operator O can control medical instrument 104 and operator assembly 106 with the perception of telepresence.

In some embodiments, medical instrument 104 may have a visualization system (discussed in more detail below), which may include a viewing scope assembly that records a concurrent or real-time image of the interventional site and provides the image to the operator or operator O through one or more displays of medical system 100, such as one or more displays of display system 110. The concurrent image may be, for example, a two-dimensional or three-dimensional image captured by an endoscope positioned within the interventional site. In some embodiments, the visualization system includes endoscopic components that may be integrally or removably coupled to medical instrument 104. However, in some embodiments, a separate endoscope, attached to a separate manipulator assembly may be used with medical instrument 104 to image the interventional site. The visualization system may be implemented as hardware, firmware, software or a combination thereof which interact with or are otherwise executed by one or more computer processors, which may include the processors of a control system 112.

Display system 110 may also display an image of the interventional site and medical instruments captured by the visualization system. In some examples, teleoperated medical system 100 may configure medical instrument 104 and controls of operator assembly 106 such that the relative positions of the medical instruments are similar to the relative positions of the eyes and hands of operator O. In this manner operator O can manipulate medical instrument 104 and the hand control as if viewing the workspace in substantially true presence. By true presence, it is meant that the presentation of an image is a true perspective image simulating the viewpoint of a physician that is physically manipulating medical instrument 104.

In some examples, display system 110 may present images of an interventional site recorded pre-operatively or intra-operatively using image data from imaging technology such as, computed tomography (CT), magnetic resonance imaging (MRI), fluoroscopy, thermography, ultrasound, optical coherence tomography (OCT), thermal imaging, impedance imaging, laser imaging, nanotube X-ray imaging, and/or the like. The pre-operative or intra-operative image data may be presented as two-dimensional, three-dimensional, or four-dimensional (including e.g., time based or velocity based information) images and/or as images from models created from the pre-operative or intra-operative image data sets.

In some embodiments, often for purposes of imaged guided surgical procedures, display system 110 may display a virtual navigational image in which the actual location of medical instrument 104 is registered (i.e., dynamically referenced) with the preoperative or concurrent images/model. This may be done to present the operator O with a virtual image of the internal interventional site from a viewpoint of medical instrument 104. In some examples, the viewpoint may be from a tip of medical instrument 104. An image of the tip of medical instrument 104 and/or other graphical or alphanumeric indicators may be superimposed on the virtual image to assist operator O controlling medical instrument 104. In some examples, medical instrument 104 may not be visible in the virtual image.

In some embodiments, display system 110 may display a virtual navigational image in which the actual location of medical instrument 104 is registered with preoperative or concurrent images to present the operator O with a virtual image of medical instrument 104 within the interventional site from an external viewpoint. An image of a portion of medical instrument 104 or other graphical or alphanumeric indicators may be superimposed on the virtual image to assist operator O in the control of medical instrument 104. As described herein, visual representations of data points may be rendered to display system 110. For example, measured data points, moved data points, registered data points, and other data points described herein may be displayed on display system 110 in a visual representation. The data points may be visually represented in a user interface by a plurality of points or dots on display system 110 or as a rendered model, such as a mesh or wire model created based on the set of data points. In some examples, the data points may be color coded according to the data they represent. In some embodiments, a visual representation may be refreshed in display system 110 after each processing operation has been implemented to alter data points.

Teleoperated medical system 100 may also include control system 112. Control system 112 includes at least one memory and at least one computer processor (not shown) for effecting control between medical instrument 104, operator assembly 106, sensor system 108, and display system 110. Control system 112 also includes programmed instructions (e.g., a non-transitory machine-readable medium storing the instructions) to implement some or all of the methods described in accordance with aspects disclosed herein, including instructions for providing information to display system 110. While control system 112 is shown as a single block in the simplified schematic of FIG. 1, the system may include two or more data processing circuits with one portion of the processing optionally being performed on or adjacent to manipulator assembly 102, another portion of the processing being performed at operator assembly 106, and/or the like. The processors of control system 112 may execute instructions comprising instruction corresponding to processes disclosed herein and described in more detail below. Any of a wide variety of centralized or distributed data processing architectures may be employed. Similarly, the programmed instructions may be implemented as a number of separate programs or subroutines, or they may be integrated into a number of other aspects of the teleoperational systems described herein. In one embodiment, control system 112 supports wireless communication protocols such as Bluetooth, IrDA, HomeRF, IEEE 802.11, DECT, and Wireless Telemetry.

In some embodiments, control system 112 may receive force and/or torque feedback from medical instrument 104. Responsive to the feedback, control system 112 may transmit signals to operator assembly 106. In some examples, control system 112 may transmit signals instructing one or more actuators of manipulator assembly 102 to move medical instrument 104. Medical instrument 104 may extend into an internal interventional site within the body of patient P via openings in the body of patient P. Any suitable conventional and/or specialized actuators may be used. In some examples, the one or more actuators may be separate from, or integrated with, manipulator assembly 102. In some embodiments, the one or more actuators and manipulator assembly 102 are provided as part of a teleoperational cart positioned adjacent to patient P and operating table T.

Control system 112 may optionally further include a virtual visualization system to provide navigation assistance to operator O when controlling medical instrument 104 during an image-guided surgical procedure. Virtual navigation using the virtual visualization system may be based upon reference to an acquired preoperative or intraoperative dataset of anatomic passageways. The virtual visualization system processes images of the interventional site imaged using imaging technology such as computerized tomography (CT), magnetic resonance imaging (MRI), fluoroscopy, thermography, ultrasound, optical coherence tomography (OCT), thermal imaging, impedance imaging, laser imaging, nanotube X-ray imaging, and/or the like. Software, which may be used in combination with manual inputs, is used to convert the recorded images into segmented two-dimensional or three-dimensional composite representation of a partial or an entire anatomic organ or anatomic region. An image data set is associated with the composite representation. The composite representation and the image data set describe the various locations and shapes of the passageways and their connectivity. The images used to generate the composite representation may be recorded preoperatively or intra-operatively during a clinical procedure. In some embodiments, a virtual visualization system may use standard representations (i.e., not patient specific) or hybrids of a standard representation and patient specific data. The composite representation and any virtual images generated by the composite representation may represent the static posture of a deformable anatomic region during one or more phases of motion (e.g., during an inspiration/expiration cycle of a lung).

During a virtual navigation procedure, sensor system 108 may be used to compute an approximate location of medical instrument 104 with respect to the anatomy of patient P. The location can be used to produce both macro-level (external) tracking images of the anatomy of patient P and virtual internal images of the anatomy of patient P. The system may implement one or more electromagnetic (EM) sensor, fiber optic sensors, and/or other sensors to register and display a medical implement together with preoperatively recorded surgical images, such as those from a virtual visualization system. For example, U.S. patent application Ser. No. 13/107,562 (filed May 13, 2011) (disclosing “Medical System Providing Dynamic Registration of a Model of an Anatomic Structure for Image-Guided Surgery”) which is incorporated by reference herein in its entirety, discloses one such system. Teleoperated medical system 100 may further include optional operations and support systems (not shown) such as illumination systems, steering control systems, irrigation systems, and/or suction systems. In some embodiments, teleoperated medical system 100 may include more than one teleoperational manipulator assembly and/or more than one operator assembly. The exact number of teleoperational manipulator assemblies will depend on the surgical procedure and the space constraints within the operating room, among other factors. Operator assembly 106 may be collocated or they may be positioned in separate locations. Multiple operator assemblies allow more than one operator to control one or more teleoperational manipulator assemblies in various combinations.

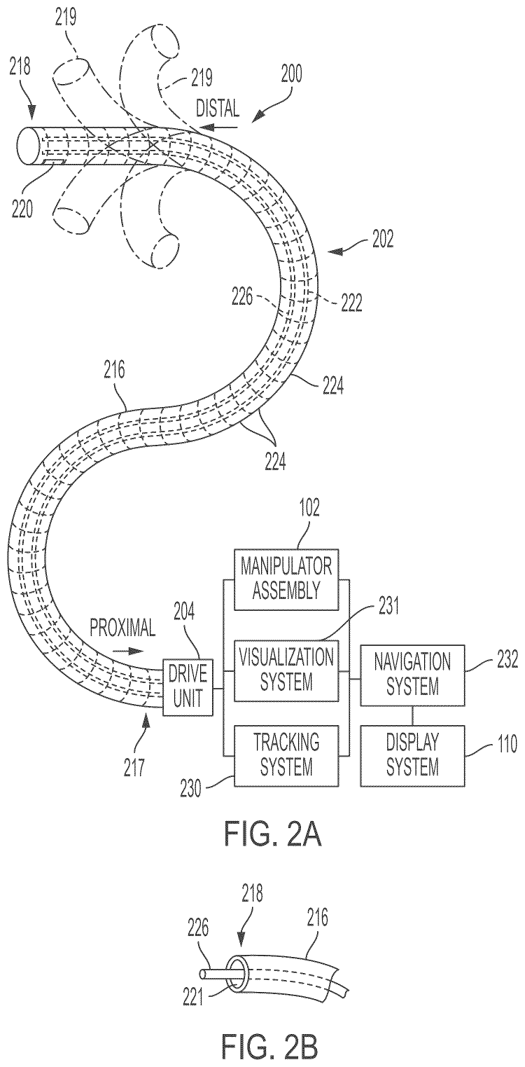

FIG. 2A is a simplified diagram of a medical instrument system 200 according to some embodiments. In some embodiments, medical instrument system 200 may be used as medical instrument 104 in an image-guided medical procedure performed with teleoperated medical system 100. In some examples, medical instrument system 200 may be used for non-teleoperational exploratory procedures or in procedures involving traditional manually operated medical instruments, such as endoscopy. Optionally medical instrument system 200 may be used to gather (i.e., measure) a set of data points corresponding to locations within anatomic passageways of a patient, such as patient P.

Medical instrument system 200 includes elongate member 202, such as a flexible catheter, coupled to a drive unit 204. In some embodiments, drive unit 204 may be coupled to or integrated within manipulator assembly 102. Elongate member 202 includes a flexible body 216 having proximal end 217 and distal end 218. In some embodiments, flexible body 216 has an approximately 3 mm outer diameter. Other flexible body outer diameters may be larger or smaller.

Medical instrument system 200 further includes a tracking system 230 for determining the position, orientation, speed, velocity, pose, and/or shape of distal end 218 and/or of one or more segments 224 along flexible body 216 using one or more sensors and/or imaging devices as described in further detail below. The entire length of flexible body 216, between distal end 218 and proximal end 217, may be effectively divided into segments 224. Tracking system 230 may optionally be implemented as hardware, firmware, software or a combination thereof which interact with or are otherwise executed by one or more computer processors, which may include the processors of control system 112 in FIG. 1.

Tracking system 230 may optionally track distal end 218 and/or one or more of the segments 224 using a shape sensor 222. Shape sensor 222 may optionally include an optical fiber aligned with flexible body 216 (e.g., provided within an interior channel or lumen (not shown) or mounted externally). In one embodiment, the optical fiber has a diameter of approximately 200 μm. In other embodiments, the dimensions may be larger or smaller. The optical fiber of shape sensor 222 forms a fiber optic bend sensor for determining the shape of flexible body 216. In one alternative, optical fibers including Fiber Bragg Gratings (FBGs) are used to provide strain measurements in structures in one or more dimensions. Various systems and methods for monitoring the shape and relative position of an optical fiber in three dimensions are described in U.S. patent application Ser. No. 11/180,389 (filed Jul. 13, 2005) (disclosing “Fiber optic position and shape sensing device and method relating thereto”); U.S. patent application Ser. No. 12/047,056 (filed on Jul. 16, 2004) (disclosing “Fiber-optic shape and relative position sensing”); and U.S. Pat. No. 6,389,187 (filed on Jun. 17, 1998) (disclosing “Optical Fibre Bend Sensor”), which are all incorporated by reference herein in their entireties. Sensors in some embodiments may employ other suitable strain sensing techniques, such as Rayleigh scattering, Raman scattering, Brillouin scattering, and Fluorescence scattering. In some embodiments, the shape of the elongate member may be determined using other techniques. For example, a history of the distal end pose of flexible body 216 can be used to reconstruct the shape of flexible body 216 over the interval of time. In some embodiments, tracking system 230 may optionally and/or additionally track distal end 218 using a position sensor system 220. Position sensor system 220 may be a component of an EM sensor system with position sensor system 220 including one or more conductive coils that may be subjected to an externally generated electromagnetic field. Each coil of the EM sensor system then produces an induced electrical signal having characteristics that depend on the position and orientation of the coil relative to the externally generated electromagnetic field. In some embodiments, position sensor system 220 may be configured and positioned to measure six degrees of freedom, e.g., three position coordinates X, Y, Z and three orientation angles indicating pitch, yaw, and roll of a base point or five degrees of freedom, e.g., three position coordinates X, Y, Z and two orientation angles indicating pitch and yaw of a base point. Further description of a position sensor system is provided in U.S. Pat. No. 6,380,732 (filed Aug. 11, 1999) (disclosing “Six-Degree of Freedom Tracking System Having a Passive Transponder on the Object Being Tracked”), which is incorporated by reference herein in its entirety.

In some embodiments, tracking system 230 may alternately and/or additionally rely on historical pose, position, or orientation data stored for a known point of an instrument system along a cycle of alternating motion, such as breathing. This stored data may be used to develop shape information about flexible body 216. In some examples, a series of positional sensors (not shown), such as electromagnetic (EM) sensors similar to the sensors in position sensor 220 may be positioned along flexible body 216 and then used for shape sensing. In some examples, a history of data from one or more of these sensors taken during a procedure may be used to represent the shape of elongate member 202, particularly if an anatomic passageway is generally static.

Flexible body 216 includes a channel 221 (FIG. 2B) sized and shaped to receive a medical instrument 226. FIG. 2B is a simplified diagram of flexible body 216 with medical instrument 226 extended according to some embodiments. In some embodiments, medical instrument 226 is a tool that may be used for procedures such as surgery, biopsy, ablation, illumination, irrigation, or suction. Medical instrument 226 can be deployed through channel 221 of flexible body 216 and used at a target location within the anatomy. Medical instrument 226 may include, for example, image capture probes, biopsy instruments, laser ablation fibers, microwave ablation tools, cryoablation tools, vapor ablation tools, and/or other surgical, diagnostic, or therapeutic tools. Medical tools may include end effectors having a single working member such as a scalpel, a blunt blade, an optical fiber, an electrode, and/or the like. Other end effectors may include, for example, forceps, graspers, scissors, clip appliers, needle drivers, retractors, stabilizers and/or the like. Other end effectors may further include electrically activated end effectors such as electrosurgical electrodes, transducers, sensors, and/or the like. Medical instrument 226 may be advanced from the opening of channel 221 to perform the procedure and then retracted back into the channel when the procedure is complete. For example, the medical instrument 226 may be an ablation tool that extends beyond the distal end 218 of the elongate member 202 to perform the ablation. In various embodiments, medical instrument 226 is a biopsy instrument, which may be used to remove sample tissue or a sampling of cells from a target anatomic location. Medical instrument 226 may be used with an image capture probe also within flexible body 216. In various embodiments, medical instrument 226 may be an image capture probe (also referred to as “image capture instrument”) that includes a distal portion with a stereoscopic or monoscopic camera at or near distal end 218 of flexible body 216 for capturing images (including video images) that are processed by a visualization system 231 for display and/or provided to tracking system 230 to support tracking of distal end 218 and/or one or more of the segments 224. The image capture probe may include a cable coupled to the camera for transmitting the captured image data. In some examples, the image capture instrument may be a fiber-optic bundle, such as a fiberscope, that couples to visualization system 231. The image capture instrument may be single or multi-spectral, for example capturing image data in one or more of the visible, infrared, and/or ultraviolet spectrums. Medical instrument 226 may be removed from proximal end 217 of flexible body 216 or from another optional instrument port (not shown) along flexible body 216.

Medical instrument 226 may additionally house cables, linkages, or other actuation controls (not shown) that extend between its proximal and distal ends to controllably the bend distal end of medical instrument 226. Steerable instruments are described in detail in U.S. Pat. No. 7,316,681 (filed on Oct. 4, 2005) (disclosing “Articulated Surgical Instrument for Performing Minimally Invasive Surgery with Enhanced Dexterity and Sensitivity”) and U.S. patent application Ser. No. 12/286,644 (filed Sep. 30, 2008) (disclosing “Passive Preload and Capstan Drive for Surgical Instruments”), which are incorporated by reference herein in their entireties. In some embodiments, medical instrument 226 may include end effectors such as those previously described above which fixed to a distal end portion of medical instrument 226 or fixed to an articulatable wrist integrated into the distal end portion of medical instrument 226. Cables, linkages, or other actuation controls may be used to control actuation of the end effector (e.g., grasping, pinching, and/or cutting actuation) or control positioning of the end effector via the articulatable wrist. Such cables, linkages, or other actuation controls may terminate in and be controlled by mechanisms within a drive unit, such as drive unit 204.

Flexible body 216 may also house cables, linkages, or other steering controls (not shown) that extend between drive unit 204 and distal end 218 to controllably bend distal end 218 as shown, for example, by broken dashed line depictions 219 of distal end 218. In some examples, at least four cables are used to provide independent “up-down” steering to control a pitch of distal end 218 and “left-right” steering to control a yaw of distal end 218. Steerable elongate members are described in detail in U.S. patent application Ser. No. 13/274,208 (filed Oct. 14, 2011) (disclosing “Catheter with Removable Vision Probe”), which is incorporated by reference herein in its entirety. In embodiments in which medical instrument system 200 is actuated by a teleoperational assembly, drive unit 204 may include drive inputs that removably couple to and receive power from drive elements, such as actuators, of the teleoperational assembly. In some embodiments, medical instrument system 200 may include gripping features, manual actuators, or other components for manually controlling the motion of medical instrument system 200. Elongate member 202 may be steerable or, alternatively, the system may be non-steerable with no integrated mechanism for operator control of the bending of distal end 218. In some examples, one or more lumens, through which medical instruments can be deployed and used at a target surgical location, are defined in the walls of flexible body 216.

In some embodiments, medical instrument system 200 may include a flexible bronchial instrument, such as a bronchoscope or bronchial catheter, for use in examination, diagnosis, biopsy, or treatment of a lung. Medical instrument system 200 is also suited for navigation and treatment of other tissues, via natural or surgically created connected passageways, in any of a variety of anatomic systems, including the colon, the intestines, the kidneys and kidney calices, the brain, the heart, the circulatory system including vasculature, and/or the like.

The information from tracking system 230 may be sent to a navigation system 232 where it is combined with information from visualization system 231 and/or the preoperatively obtained models to provide the physician or other operator with real-time position information. In some examples, the real-time position information may be displayed on display system 110 of FIG. 1 for use in the control of medical instrument system 200. In some examples, control system 116 of FIG. 1 may utilize the position information as feedback for positioning medical instrument system 200. Various systems for using fiber optic sensors to register and display a surgical instrument with surgical images are provided in U.S. patent application Ser. No. 13/107,562, filed May 13, 2011, disclosing, “Medical System Providing Dynamic Registration of a Model of an Anatomic Structure for Image-Guided Surgery,” which is incorporated by reference herein in its entirety.

In some examples, medical instrument system 200 may be teleoperated within medical system 100 of FIG. 1. In some embodiments, manipulator assembly 102 of FIG. 1 may be replaced by direct operator control. In some examples, the direct operator control may include various handles and operator interfaces for hand-held operation of the instrument.

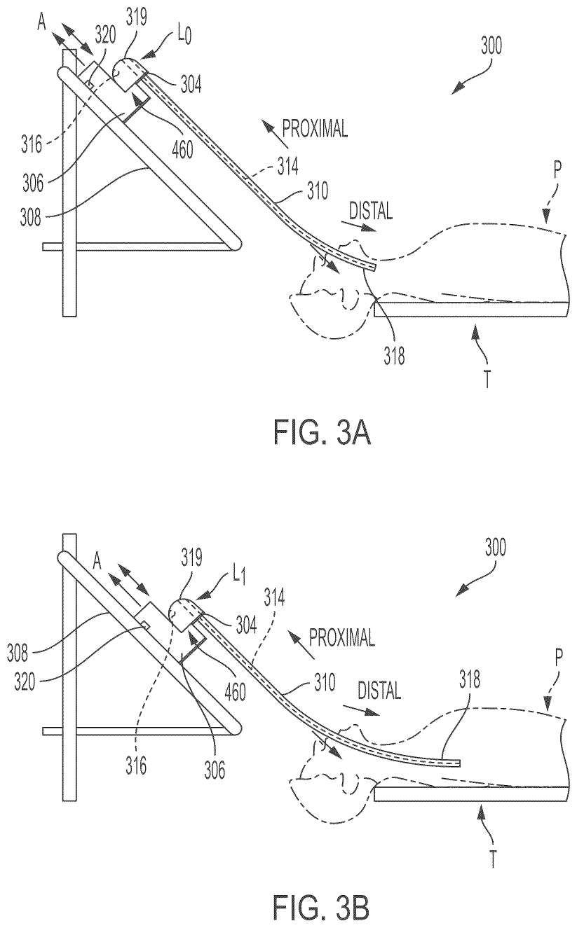

FIGS. 3A and 3B are simplified diagrams of side views of a medical instrument mounted on an insertion assembly and position to treat a patient according to some embodiments. As shown in FIGS. 3A and 3B, a surgical environment 300 includes a patient P positioned on the table T of FIG. 1. Patient P may be stationary within the surgical environment in the sense that gross patient movement is limited by sedation, restraint, and/or other means. Cyclic anatomic motion including respiration and cardiac motion of patient P may continue, unless patient is asked to hold his or her breath to temporarily suspend respiratory motion. Accordingly, in some embodiments, data may be gathered at a specific, phase in respiration, and tagged and identified with that phase. In some embodiments, the phase during which data is collected may be inferred from physiological information collected from patient P. Within surgical environment 300, a backend mechanism 304 can be removably coupled to an instrument carriage 306. In some embodiments, the backend mechanism 304 may be formed of a housing containing operational components for cables used to provide independent up down steering to control a pitch of distal end 318 and left right steering to control a yaw of distal end 318. In addition, the backend mechanism 304 may comprise EM sensors, shape-sensors, and/or other sensor modalities and or provide connectors coupling sensing modalities to an instrument such as elongate member 310.

Instrument carriage 306 can be mounted to an insertion stage 308 which is fixed within surgical environment 300. Alternatively, insertion stage 308 may be movable but have a known location (e.g., via a tracking sensor or other tracking device) within surgical environment 300. Instrument carriage 306 may be a component of a teleoperational manipulator or a non-teleoperational manipulator assembly (e.g., manipulator assembly 102) that controls insertion motion (i.e., motion along the A axis) and, optionally, motion of a distal end 318 of an elongate member 310 in multiple directions including yaw, pitch, and roll. Instrument carriage 306 or insertion stage 308 may include actuators, such as servomotors, (not shown) that control motion of instrument carriage 306 along insertion stage 308, control motion of the distal end 318 of elongate member 310 in yaw/pitch, and/or control roll motion of elongate member 310 along a longitudinal axis. In some embodiments described herein, the actuators used to control movement of the elongated member attached to the carriage or other appropriate structure, may be referred to as elongate member actuators.

Elongate member 310 is coupled to backend mechanism 304. Backend mechanism 304 is coupled and fixed relative to instrument carriage 306. In some embodiments, an optical fiber shape sensor 314 is fixed at a proximal point 316 on backend mechanism 304. In some embodiments, proximal point 316 of optical fiber shape sensor 314 may be movable along with backend mechanism 304 but the location of proximal point 316 may be known (e.g., via a tracking sensor or other tracking device). Shape sensor 314 measures a shape from proximal point 316 to another point such as distal end 318 or a point along a distal portion of elongate member 310.

A position measuring device 320 provides information about the position of backend mechanism 304 as it moves on insertion stage 308 along an insertion axis A. Position measuring device 320 may include resolvers, encoders, potentiometers, and/or other sensors that determine the rotation and/or orientation of the actuators controlling the motion of instrument carriage 306 and consequently the motion of backend mechanism 304. In some embodiments, insertion stage 308 is linear. In some embodiments, insertion stage 308 may be curved or have a combination of curved and linear sections.

FIG. 3A shows backend mechanism 304 and instrument carriage 306 in a retracted position along insertion stage 308. In this retracted position, proximal point 316 is at a position L₀ on axis A. In this position along insertion stage 308 a component of the location of proximal point 316 may be set to a zero and/or another reference value to provide a base reference to describe the position of instrument carriage 306, and thus proximal point 316, on insertion stage 308. With this retracted position of backend mechanism 304 and instrument carriage 306, distal end 318 of elongate member 310 may be positioned proximal to, e.g., just inside, just outside, or otherwise near an entry orifice of patient P. Also in this position, position measuring device 320 may be set to a zero and/or another reference value (e.g., I=0). In FIG. 3B, backend mechanism 304 and instrument carriage 306 have advanced along the linear track of insertion stage 308 and distal end 318 of elongate member 310 has advanced into patient P. In this advanced position, the proximal point 316 is at a position L₁ on the axis A. In some examples, encoder and/or other position data from one or more actuators controlling movement of instrument carriage 306 along insertion stage 308 and/or one or more position sensors associated with instrument carriage 306 and/or insertion stage 308 is used to determine the position Lₓ of proximal point 316 relative to position L₀. In some examples, position Lₓ may further be used as an indicator of the distance or insertion depth to which distal end 318 of elongate member 310 is inserted into the passageways of the anatomy of patient P.

In FIG. 3A, the backend mechanism 304 includes a mounting face 460, that may define a mounting plane. A portion of the mounting face 460, referred to as an interfacing region, is disposed against the instrument carriage 306, while another portion of the mounting face 460, referred to as a non-interfacing region, protrudes outwardly beyond an edge of the instrument carriage 306. As can be seen, the elongate member 310 extends from the backend mechanism 304, out of the mounting face 460, and past the instrument carriage 306.

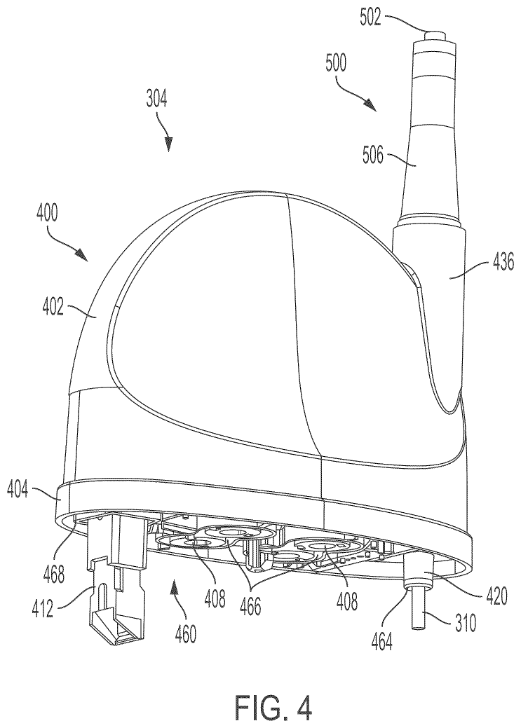

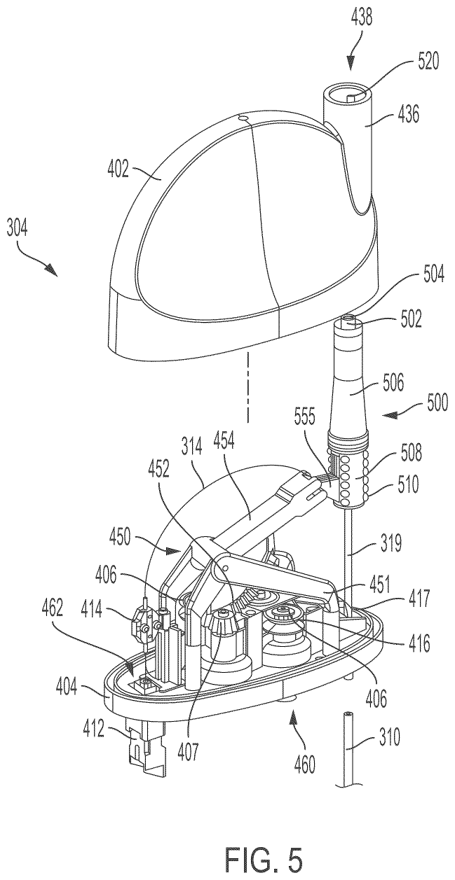

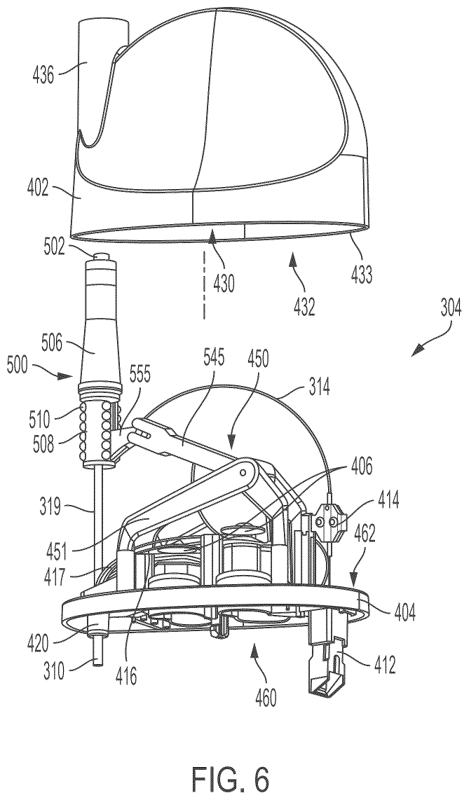

FIGS. 4, 5, and 6 are perspective views of the backend mechanism 304 independent of the instrument carriage 306 from FIGS. 3A and 3B, and including a proximal portion 319 of the elongate member 310. FIG. 4 shows the backend mechanism 304 in an assembled state, and FIGS. 5 and 6 show the backend mechanism 304 in a partially exploded state. Referring to these Figures, the backend mechanism 304 includes the housing 400, including a cover 402 and a chassis 404. The chassis 404 carries a plurality of steering components 406, a plurality of drive components 408, a tool support fixture 450, a fiber connector 412, and a launch region fixture 414. The proximal portion 319 of the elongate member 310 extends through and terminates within the housing 400. FIG. 5 shows the shape sensor 314 extending from the proximal portion 319 of the elongate member 310 to the launch region fixture 414 and a plurality of coil pipes 417 with pull wires 416 disposed therein extending from the elongate member. The launch region fixture 414 is comprised of a series of stabilizing components arranged to rigidly support the proximal end of the shape sensor 314. The coil pipes 417 and the pull wires 416 also extend from the elongate member 310 to the plurality of steering components 406. The housing 400, including the cover 402 and the chassis 404, is selectively attachable to the instrument carriage 306 (shown in FIGS. 3A and 3B) and provides a compact manageable unit that securely protects steering and sensing components from the surgical environment. The pull wire 416 may extend through a lumen in the coil pipe 417, extend from the terminal proximal end of the coil pipe 417, and route around steering component 406 and drive component 408. Various components of the backend mechanism 304, including the steering components 406 and drive components 408 are further described in U.S. patent application Ser. No. 17/047,414, which is incorporated by reference herein in its entirety. Additionally, it should be understood that while particular steering, drive components, and shape sensor are described in relation to the figures, the current disclosure is not limited to the use of these specific components.

The cover 402 is shown in FIGS. 4-6. The cover 402 may comprise a cavity 430 sized and arranged to cover and protect the steering components 406, the drive components 408, and other components carried by the chassis 404. An opening 432 to the cavity 430 is defined by edges 433 shaped to interface with the chassis 404. In the implementation shown, the cover 402 comprises a protruding boss 436 extending in a direction opposite the opening 432. In this implementation, the protruding boss 436 comprises a passage 438 providing access to the elongate member 310 and through which the elongate member 310 may at least partially extend. In some implementations, the cover 402 may serve as a handler gripping surface for the backend mechanism 304. Accordingly, it may be shaped and sized for convenient grasping by a human hand in some embodiments.

The chassis 404 may be arranged to support components of the backend mechanism 304. For example, the chassis 404 may support the steering components 406, drive components 408, the tool support fixture 450, the fiber connector 412, and the launch region fixture 414. The chassis 404 may include a mounting face 460 and an opposing components support face 462. A plurality of openings extends through the chassis 404 from the components support face 462 to the mounting face 460. For example, the chassis 404 includes an elongate member opening 464, drive component openings 466, and a fiber connector opening 468. The elongate member 310 extends through the elongate member opening 464, the drive components 408 extend through the drive component openings 466, and the fiber connector 412 extends through the fiber connector opening 468. These openings may be used to provide electrical or mechanical connection between components forming an outer portion of the backend mechanism 304 and components disposed within the housing 400 of the backend mechanism 304. The mounting face 460 may be arranged to interface with the instrument carriage 306. In some implementations, the instrument carriage 306 may include drive mechanisms such as drive motors, which interface with and drive the drive components 408 of the backend mechanism 304 and may include pin or other connectors that may provide an electrical communication interface that may interface with a printed circuit assembly, for example.

In some embodiments, an elongate member 310 may be axially fixed to the housing 400 of the backend mechanism. In some embodiments, the elongate member may be fixed within opening 464 of the chassis 404. As shown in FIGS. 4 and 6, the chassis 404 may include a boss 420 to engage and retain the elongate member 310.

As shown in FIGS. 4 to 6, the fiber connector 412 projects from the mounting face 460 of the chassis 404. A matching receiving connector (not shown) is disposed on the instrument carriage 306. Fiber connector 412 communicates information from the shape sensor 314 to the instrument carriage 306 and ultimately to the control system 112

The steering components 406 are arranged to direct the pull wires 416 that extend from the elongate member 310 to the drive components 408. The pull wires 416 may be axially tightened or loosened to displace the distal end 318 of the elongate member 310 as described above. Each steering component 406 may include a pulley including a wheel and an axle. Here, the axle defines the axis about which the wheel rotates. The wheel may be formed of a low friction pulley material to enable free rotation about the axle. In some implementations, the low friction pulley material is a metal, such as stainless steel or aluminum with a low friction bearing, while in other implementations, the low friction pulley material is a polymer material such as, without limitation, polyethylene terephthalate (PET), acetal (POM), polyamides, and others, all of which may optionally be enhanced by compounded or applied lubricants such as PTFE, silicone oil, paraffin wax and others. The wheel may include a deep, v-shaped pull wire supporting surface configured to recapture slack loop in the pull wires including when the slack loop deviates from the pulley groove centerline beyond the outer wall of the pulley wheel. In addition, the groove may be wide to assist in capture. In some implementations, slack in the pull wires may be created during bending of the elongate member 310. The width of the groove may re-direct the pull wire onto the wheel even if the slack temporarily removes the pull wire from the groove. In some implementations, the pull wire may have a bend that causes lateral displacement when the pull wire has slack. The wide groove may assist with recapture of the pull wire into the groove. The groove may be aligned with a tangent reference line intersecting with the axis of the elongate member 310 or may be offset from it. Of course, while a specific actuator arrangement with steering components 406 is shown in the figures, any other appropriate type of actuator arrangement for controlling actuation and/or articulation of the system may be used.

The drive components 408 may interface with the pull wires 416 and may be driven by motors on the instrument carriage 306. Accordingly, the drive components 408 may increase and decrease tension in the pull wires 416 to effect movement at the distal end 318 of the elongate member 310.

In implementations employing three pull wires 416, each pull wire may be fixed to and extend from a distal end of the elongate member 310, separated along the circumference of the elongate member by an appropriate angle (e.g., 120°). In alternative implementations, any number of pull wires may be employed, each extending from the distal end of the elongate member 310, and spaced apart at varying distances along the circumference of the elongate member depending on the desired steering configuration. In some implementations, each pull wire 416 runs through the coil pipe 417 which is coupled to a distal section of the elongate member 310. Each coil pipe 417 may be paired with a pull wire 416 and extend the length of the elongate member to a distal portion of the elongate member, and exit the elongate member at a proximal portion. An example of pull wires and coil pipes in the elongate member may be found in U.S. Provisional Patent 62/535,673, filed on Jul. 21, 2017, titled “Flexible Elongate Devices Systems and Methods,” which is incorporated herein by reference. In some implementations, the coil pipes, and the pull wires disposed therein, exit the elongate member 310 of the backend mechanism 304 and travel and attach to a portion of a drive component 408. However, while a particular construction is disclosed for controlling articulation of the elongate member, it should be understood that any appropriate control system for controlling articulation of the elongate member may be used.

As shown in FIGS. 4-6, in some embodiments, the chassis 404 may support up to three drive components 408. In embodiments utilizing three pull wires 416 (not shown), each drive component 408 may cooperate with a steering component 406 to direct one of the three pull wires to steer the distal end of elongate member 310. In some embodiments, one of the drive components 408 may be used to control the position of the medical tool as well. For example, in the embodiment shown in FIGS. 4-6, three drive components 408 each cooperate with a steering component 406 to direct three pull wires 416, while a fourth drive component 408 cooperates with a tool driving component 407 configured to move a tool holder 500, as described below. It should be noted that the backend mechanism may be adapted to utilize the drive mechanisms in any arrangement to suit the needs of a medical procedure or other application. The chassis 404 may support various numbers of drive components 408 and steering components 406, each providing a degree of freedom for (e.g., translational, rotational, etc.) movement of the elongate member 310 or a tool that is inserted within the elongate member 310.

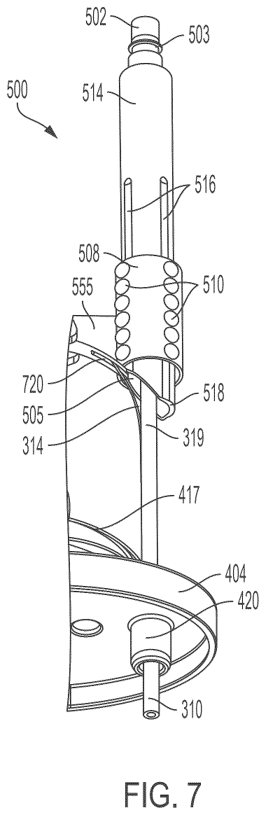

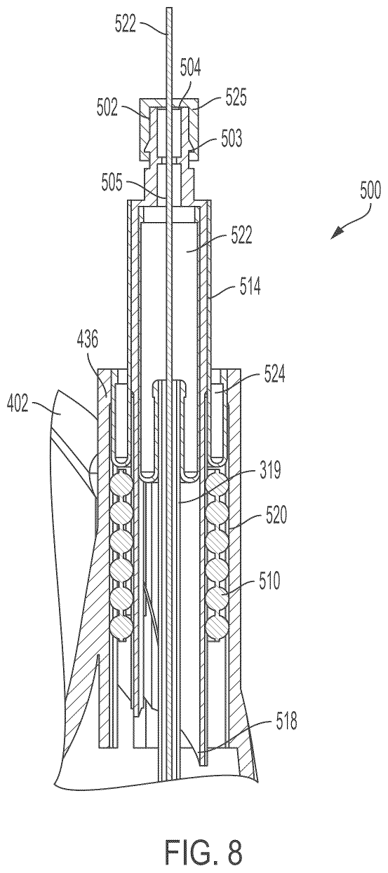

As shown in FIGS. 5-8, in some embodiments, the backend mechanism 304 may include a tool holder 500 that is moveably mounted to the housing 400 which may include the cover 402 and chassis 404. FIGS. 5 and 6 show the tool holder 500 moveably fixed to the backend mechanism 304 via a tool support fixture 450 connected to the chassis 404 or other appropriate portion of the backend mechanism 304. FIG. 7 shows an enlarged view of the tool holder 500 with a seal 506 removed, and FIG. 8 shows a cross sectional schematic of the tool holder 500.

In some embodiments, the tool holder 500 is configured to retain and lock a medical tool 522, see FIG. 8, that is inserted through the tool holder 500 and into a channel that extends through the elongate member 310. As shown in FIGS. 7 and 8, the tool holder 500 includes a tubular body 514 having a channel 505 that extends through the tubular body. A proximal portion 319 of the elongate member 310 may be at least partially received in the channel 505 and extend distally out from a distal end 518 of the tubular body 514. As noted previously, the elongate member 310 may be held stationary relative to the housing and overall backend mechanism 304 while the tool holder 500, including the tubular body 514 of the tool holder 500, may be moved relative to the housing 400 and overall backend mechanism 304.

As shown in FIGS. 5, 7 and 8, the proximal end portion 502 of the tool holder 500 includes an opening 504 to channel 505 that extends through the tool holder 500 and may be aligned with a proximal end portion 319 of the elongate member 310. A medical tool 522 may be inserted into the opening 504 and through the tool holder 500 into the channel of the elongate member 310. In some embodiments, a proximal end portion 502 of the tool holder 500 may include a locking connector 503 that may be engaged with the medical tool 522 itself or a corresponding locking connector 525 disposed on the medical tool to lock the medical tool 522 to the tool holder 500. In some embodiments, the locking connector 525 may include a tube fitting, but the disclosure is not so limited, and any locking technique may be used (e.g., compression fittings, detents, clamps). As described below, movement of the tool holder 500 causes the medical tool 522 to move relative to the elongate member 310.

Returning to FIGS. 5 and 6, in some embodiments, the tool holder 500 may be arranged to extend through a passage 438 of a protruding boss 436 of the housing 400. In some embodiments, the tool holder 500 may include a seal 506 through which the tool holder 500 extends and which is arranged to connect to an opening of the protruding boss 436. In some embodiments the seal 506 may securely attach to the opening via corresponding threads, compression fit, or any other attachment techniques. Seal 506 may be a rolling seal that is fixed on one end to the tubular body 514 and on the other to the protruding boss 436 of the cover 402. A proximal end portion 502 of the tool holder 500 may extend from a proximal opening of the seal 506 in some embodiments. The tool holder 500 may move relative to the protruding boss 436 such that a varying length of the proximal end portion 502 may extend from the seal 506 depending on the position of the tool holder 500. While a specific arrangement of the tool holder 500 relative to the housing of the backend mechanism is shown, it should be understood that any arrangement of the housing and/or tool holder 500 may be used.

As shown in FIGS. 5 and 6, the tool holder 500 may be moveably fixed to the chassis 404 via a tool support fixture 450. In some embodiments, the tool support fixture 450 may include one or more support legs 451, and a rocker arm 452 coupled to a tool arm 454. The rocker arm 452 may be rotationally coupled to the support legs 451 via a hinge such that the rocker arm 452 may rotate between the support legs 451 along a plane perpendicular to a plane of the chassis 404. The rocker arm 452 may include a plurality of teeth arranged along an outer periphery configured to engage a plurality of teeth arranged around the outer circumference of a tool driving component 407 attached to a drive component 408. The plurality of teeth on the rocker arm and on the tool driving component may act as a gear such that rotation of the drive component 408 causes the rocker arm to rotate, which rotates the tool arm 454 to translate the tool holder 500 in a direction parallel to a longitudinal axis of an adjacent portion of the elongate member 310. The drive component 408 may be appropriately controlled to control the amount of rotation of the rocker arm and corresponding translation of the tool arm 454.

As depicted in the figures, in some embodiments, the tool arm 545 is connected to an extension arm 555 of the tool holder 500 via a camming connection where a pin connected to the tool holder 500 is received in a groove formed in an adjacent portion of the rocker arm. For example, in some embodiments, a protruding arm 555 extends from an outer surface of the tubular body 514 to connect to the tool arm 454 to provide the noted camming connection. Accordingly, when the tool arm 454 rotates in a first direction (e.g., away from the chassis 404), the tool arm 454 causes the tool holder 500 to move axially within the protruding boss 436 in a direction away from the chassis while the cammed connection accommodates motion of the pin in a transverse direction as well. Similarly, when the tool arm 454 rotates in a second direction, opposite the first direction, the tool arm 454 causes the tool holder 500 to move axially within the protruding boss 436 in a direction toward the chassis. Accordingly, the drive component 408 may control the movement of the tool holder 500 and movement of medical tool attached to the tool holder 500 relative to the elongate member 310.

The embodiments described and shown in FIGS. 5 and 6 are only by way of example, and any arrangement of moveably mounting the tool holder 500 to the housing may be used. For example, any structure that converts motion of the drive component (whether that is translational or rotational motion) into motion of the tool holder 500 as well as any appropriate connection between the tool holder 500 and drive component may be incorporated into the backend mechanism (e.g., rack and pinion, tether and pulleys, direct actuation by a connected linear actuator, etc.). Additionally, any appropriate type of tool drive component, which may also be referred to as a tool actuator, may be used to drive movement of the tool holder 500 including for example, rotational motors and linear actuators.

As shown in FIGS. 5-7, in some embodiments, the tool holder 500 includes a bearing 508, such as a linear bearing, or other appropriate bearing, that extends at least partially around the outer surface of the tubular body 514 along a length of the tubular body. The bearing 508 may include a plurality of roller balls 510 arranged in one or more (e.g., at least two) rows in a direction along a longitudinal length of the tool holder 500. The tubular body may include channels 516 arranged on a longitudinal length of the tubular body. The bearing may be positioned on the tubular body 514 such that the one or more rows of roller balls 510 are positioned along and at least partially contained within the channels 516 of the tubular body. When the cover 402 of the housing is attached to the chassis 404, the one or more rows of roller balls 510 may be positioned within corresponding channels 520 that extend at least partially along an inner surface of the protruding boss 436 or other bearing surface of the housing. The roller balls 510 within channels 516 of the tubular body 514 and channels 520 of the protruding boss 436 may facilitate axial movement of the tool holder 500 within the protruding boss 436. As shown in FIGS. 5-7, the bearing 508 may not extend fully around the outer surface of the tubular body 514, leaving a gap through which the protruding arm 555 may move when the tool holder 500 moves axially within the protruding boss 436. The protruding arm 555 may also include a slot 720 through which the shape sensor 314 extends to the launch region fixture 414.

FIG. 8 shows a cross-sectional schematic view of a tool holder 500 extending through a protruding boss 436 of a cover 402 of housing 400 when the cover 402 is attached to the chassis 404. As shown, a proximal end portion 319 of an elongate member 310 extends at least partially into a distal portion of channel 505 of the tubular body 514. As shown in FIG. 8, the tool holder 500 may include one or more seals (e.g., o-ring, rolling diaphragm seal, etc.) to seal the tool channel 505 and proximal end 319 of the elongate member 310 relative to a surrounding environment. For example, the tool holder 500 may include a seal 522 (e.g., rolling diaphragm seal) arranged between an inner surface of the tubular body 514 and a proximal end 319 of the elongate member. In some embodiments, the tool holder 500 may also include one or more seals 524 (e.g., o-rings, roller diaphragm seal, etc.) arranged between an outer surface of the tubular body 514 and an inner surface of the protruding boss 436. Providing seals between the components of the housing, tool holder 500, and elongate member prevent contamination of the medical instrument during a procedure. In the above embodiment, the depicted rolling diaphragm seals may be advantageous in that they may offer a seal that easily translates with the tool holder 500 though other seals capable of accommodating the desired relative movement of the tool holder 500 may also be used.

In FIG. 8, the tool holder 500 may be in a fully retracted position, according to some embodiments. During operation, the tool holder 500 may maintain a fixed position (e.g., fully or partially retracted) relative to the housing while the medical system is in a “drive mode,” or the system controls one or more of the drive components 408 and steering components 406 (see FIGS. 4-6) to steer a distal end portion of the elongate member 310 to a desired position (e.g., proximate to target tissue). Although not shown in FIG. 8, the elongate member 310 may be axially fixed to the housing and is advanced into a patient by moving the backend mechanism toward a patient (see FIGS. 3A-3B).

In some embodiments, the medical system operates in a “drive mode” to bring the distal end portion of the elongate member to an interventional site within a patient's body. An imaging tool may be first inserted within the channel of the elongate member to capture images of the anatomical passageways of the patient that are traversed to reach the interventional site. When the elongate member 310 is properly positioned in the patient, the imaging tool may be removed from the channel of the elongate member and a medical tool 522 (e.g., ablation tool) may be inserted into the channel of the elongate member 310, such as via opening 504 of the tool holder 500 and through the channel 505 of the tool holder 500 and into the channel of the elongate member 310 until a distal end portion of the medical tool reaches the distal end portion of the elongate member 310. In another example, the medical tool 522 is inserted into the elongate member 310 and then the distal end portion of the elongate member is brought to an interventional site. The medical tool, including the distal end portion, may remain within the channel of the elongate member until the medical system is changed from the “drive mode” to an “expose mode.” In the “expose mode,” the medical system may simultaneously retract the elongate member while extending the medical tool relative to the elongate member to expose the distal end portion of the medical tool and maintain the position and orientation of the medical tool 522 to facilitate operation of the medical tool 522 at the interventional site.

In the “expose mode,” the medical system may automatically coordinate the movement of the elongate member (e.g., via movement of the backend mechanism 304) As described above with respect to FIGS. 3A-3B, a position measuring device 320 may provide information about the position of backend mechanism and may determine the rotation and/or orientation of an actuator controlling the motion of the backend mechanism (e.g., via instrument carriage 306). In some embodiments, a positioning sensor, such as an encoder of the drive components, a displacement sensor, and/or any other appropriate type of sensor may determine the position of tool holder 500 relative to the reference frame of the backend mechanism. Based on the positional data from the positioning sensors, the medical system may control the actuators to move the backend mechanism and the tool holder 500 to coordinate the steps in “expose mode.” Referring to FIG. 8, during “expose mode,” the tool holder 500, including the tubular body 514, will move axially in a distal direction toward the elongate member 310 while the overall backend mechanism is moved in a proximal direction. In embodiments where these two movements are equal in magnitude the distal end portion of the medical tool 522 may be extended out from the distal end of the elongate member 310 while maintaining the pose of the medical tool 522. In some embodiments, there may be a delay between movement of the backend mechanism and movement of the tip of the elongate member 310 in the “expose mode.” For example, initial retractive movement of the backend mechanism does not necessarily cause movement at the distal tip of the elongate member 310. There may be slack in the elongate member 310 that is within the passageway of the anatomy and the initial movement may only result in the elongate member 310 becoming more taught within the passageway. The distal tip only moves after there is no remaining slack in the elongate member 310. A sensor (e.g., the shape sensor 314 or some other position sensor) may be used to detect the movement of the distal tip of the elongate member 310, including while steering the elongate member 310 during retraction (e.g., in order to eliminate slack in the elongate member). The control system may initiate movement of the medical tool 522 relative to the elongate member 310 (e.g., insertion of the medical tool 522) in response to detecting the movement of the distal tip of the elongate member 310 using the sensor (e.g., rather than in connection with actuation of the backend mechanism).

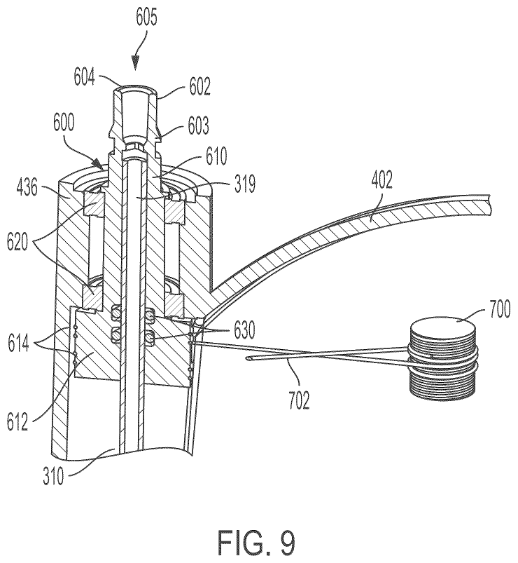

FIG. 9 is a cross sectional schematic of another embodiment of a tool holder 600 of a backend mechanism that may rotate a medical tool (not shown) retained in the tool holder 600 relative to an elongate member 310. As shown in FIG. 9, the tool holder 600 may include an elongated tubular body 610 with a channel 605 that extends therethrough. In some embodiments, the tool holder 600 may be arranged within and at least partially extend from a protruding boss 436 of a housing cover 402. A proximal end 319 of an elongate member 310 may be received within the channel 605 of the tool holder 600 and extend from a distal end portion 612 of the tool holder 600. The elongate member 310 may be axially and/or rotationally fixed to a portion of the backend mechanism housing such that the elongate member remains in a fixed position relative to the backend mechanism.

In some embodiments, the backend mechanism may include a rotational drive component 700 that is operatively coupled to a drive component 408 (see FIGS. 5-6) such that rotation of the drive component 408 causes rotation of the rotational drive component 700. A pull wire 702 may connect the rotational drive component 700 to the distal end portion 612 of the tool driver, translating rotational motion of the rotational drive component 700 to rotational motion of the tool holder 600 and an associated tool, not shown. It should be noted that a backend mechanism may include a rotational drive component 700 in additional to a tool driving component 407 configured to impart translational movement to the tool holder (see FIGS. 5-6). For example, a backend mechanism may include two pull wires connected to two drive components to control the steering of an elongate member, providing two drive components to couple to a rotational drive component and a tool driving component. The drive components may be utilized in any configurations.

Similar to the above translational system, in some embodiments, the tool holder 600 may include one or more bearings 614 around an outer surface of the tool holder 600 to facilitate rotation (and/or translation) of the tool holder 600 relative to the protruding boss. In some embodiments, the tool holder 600 may include one or more seals or bearing 620 to maintain the integrity of backend mechanism. For example, seals 620 may be arranged between an inner surface of the protruding boss 436 and an outer surface of tubular body 610 of the tool holder 600. Seals 630 may be arranged between an inner surface of the elongated tubular body 610 of the tool holder 600 and the proximal portion 319 of the elongate member 310. Seals 630 keeps any fluid that may enter the proximal portion 319 of the elongate member 310 from the proximal end portion 602 goes to elongate member 310 and not the backend mechanism.

In some embodiments, a medical tool (not shown) nay be inserted through opening 604 of the tool holder 600 into channel 605 of the tool holder 600 and through a channel of the elongate member 310. The tool holder 600 may have a proximal end portion 602 that includes a locking connection 603 that retains and locks the medical tool to the tool holder 600. When the rotational drive component 700 rotates the tool holder 600, the tool holder 600 consequently rotates that medical tool relative to the elongate member. A positional sensor may provide data about the orientation and/or rotational position of the tool holder 600. Based on this data, the medical system may direct drive components to translate or rotate the tool holder 600 relative to the housing to control a position of the medical tool.

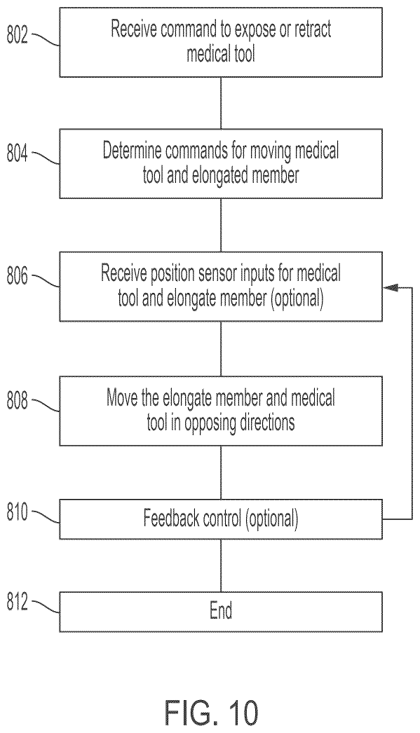

FIG. 10 is one embodiment of a method that may be used to control the position of the elongate member and a medical tool in a medical instrument. In some embodiments, the method may be implemented using a controller including one or more processors associated with memory including computer executable instructions. Specifically, when executed, the controller may control any of the disclosed systems and components herein to implement the described method. Additionally, while the steps of the depicted method are shown in a particular order, it should be understood that certain steps may be performed in different orders than a shown in the figure, may be performed simultaneously, and/or method may include one or more additional intermediate steps not shown.

In the depicted embodiment, the method may include a step 802 where a command may be received to expose or retract a medical tool relative to an elongated member of a medical instrument. For example, a user may input a command through any appropriate user interface including, for example a button, keyboard, or touchscreen. Upon receipt of the command, a controller of the system may determine one or more commands for moving the medical tool and elongated member relative to one another. For example, in instances where the tool is to be exposed, the commands may be determined to move the medical tool to a commanded distal position and the elongated member, or structure the elongated member is attached to (e.g., the disclosed backend mechanism) may be commanded to move to a commanded proximal position. In some embodiments, the commanded displacements may have magnitudes that are approximately equal to one another but in opposite directions during exposure and/or retraction of the medical tool in some embodiments.

At step 804, in some embodiments, during “drive mode,” the distal end of the elongate member is navigated until it has a desired pose (e.g., position and orientation) with respect to a target tissue for treatment. During “expose mode,” the elongate member when retracted may be articulated as needed to maintain the pose for the distal end of the medical tool. When the medical tool is an ablation tool, the relative retraction of the elongate member exposes the energy producing portion of the ablation tool to the tissue and prevents the ablation tool from damaging the elongate member during operation. Furthermore, by maintaining the pose of the ablation tool, the ablation tool is in a desired position for application of treatment to the target tissue. The ablation tool may be distally advanced from the pose of the as needed to reach the target tissue. This movement may be performed via moving the ablation tool with the elongate member (e.g., via movement of insertion stage 308), or via movement of just the ablation tool (e.g., via movement of tool holder 500). In some embodiments, the medical tool may also be rotated with respect to the elongate member (e.g., via rotational drive component 700).

In instances where the medical tool is to be retracted, the above directions for the commanded positions may be reversed to move the tool in the opposite direction. For example, the medical tool may be commanded to move in a proximal direction and the elongate member may be commanded to move in an opposing distal direction. In another example, to retract the medical tool into the channel of the elongate member, the medical tool may be commanded to move in a proximal direction while the elongate member does not move, or the elongate member may be commanded to move in a distal direction while moving the medical tool in a proximal direction more than the amount of movement of the elongate member in the distal direction.

At step 806, the controller of a medical system may optionally receive position sensor inputs for the medical tool and the elongate member prior to moving the elongate member and the medical tool in the commanded opposing directions at 808. In some instances, an appropriate feedback control, such as a closed-loop feedback control loop, may optionally be implemented at step 810 using the sensed positions of the medical tool and elongated member. Depending on the desired functionality, the feedback loop may be configured to maintain a position or pose of the medical tool substantially stationary during the commanded movement to either expose or retract the medical tool relative to the elongate member. Once the commanded movement is finished, the method may end at 812.

While the present teachings have been described in conjunction with various embodiments and examples, it is not intended that the present teachings be limited to such embodiments or examples. On the contrary, the present teachings encompass various alternatives, modifications, and equivalents, as will be appreciated by those of skill in the art. Accordingly, the foregoing description and drawings are by way of example only.

1. A medical instrument comprising:
a housing;
an elongate member axially fixed to and extending distally from the housing, the elongate member including a channel extending through the elongate member; and
a tool holder moveably mounted to the housing and configured to accept and retain a tool inserted through the tool holder and into the channel of the elongate member, wherein movement of the tool holder moves the tool relative to the elongate member.

2. The medical instrument of claim 1, wherein the elongate member includes an articulable portion extending along at least a portion of a length of the elongate member.

3. The medical instrument of claim 1, further comprising a tool actuator disposed in the housing and configured to move the tool holder between a proximal position and a distal position to move the tool relative to the elongate member.

4. The medical instrument of claim 3, further comprising a rocker arm operatively coupling the tool actuator with the tool holder.

5. The medical instrument of claim 3, further comprising an elongate member actuator configured to control the position of the elongate member.

6. The medical instrument of claim 5, wherein the elongate member actuator is configured to control a position of the housing to control the position of the elongate member.

7. The medical instrument of claim 5, further comprising at least one processor operatively coupled to the tool actuator and the elongate member actuator, wherein the at least one processor is configured to control the tool actuator to control movement of the tool and to control movement of the elongate member actuator to control movement of the elongate member.

8. The medical instrument of claim 7, wherein:
the elongate member includes a sensor configured to detect movement of a distal tip of the elongate member; and
the at least one processor is configured to, in response to detecting movement of the distal tip of the elongate member using the sensor, control the tool actuator to move the tool relative to the elongate member.

9. The medical instrument of claim 7, wherein the at least one processor is configured to control the tool actuator and the elongate member actuator to maintain a position of a distal portion of the tool during a relative movement of the tool and the elongate member that exposes the distal portion of the tool.

10. The medical instrument of claim 7, wherein the at least one processor is configured to maintain a pose of a distal portion of the tool during a relative movement of the tool and the elongate member that exposes a distal portion of the tool.

11. The medical instrument of claim 7, wherein:
the tool is an ablation tool; and
at least one processor is configured to control the ablation tool to treat a target tissue after a relative movement of the tool and the elongate member that exposes a distal portion of the ablation tool.

12. The medical instrument of claim 1, further comprising:
a first seal disposed between a portion of the tool holder and a portion of the housing; and
a second seal disposed between the elongate member and the tool holder.

13. The medical instrument of claim 1, wherein the tool holder is configured to translate relative to the housing and the elongate member.

14. The medical instrument of claim 1, wherein the tool holder is configured to rotate relative to the housing and the elongate member.

15. A method for controlling movement of a medical instrument, the method comprising:
positioning and retaining a tool in a tool holder moveably mounted to a housing, wherein the tool extends into a channel extending through an elongate member of the medical instrument; and
moving the tool holder relative to the housing while maintaining an axial position of the elongate member relative to the housing to move the tool relative to the elongate member.

16. The method of claim 15, further comprising articulating at least a portion of a length of the elongate member.

17. The method of claim 15, further comprising moving the tool holder between a proximal position and a distal position via a tool actuator disposed in the housing to move the tool relative to the elongate member.

18. The method of claim 17, further comprising coupling the tool actuator with the tool holder via a rocker arm.

19. The method of claim 17, further comprising controlling one selected from a group consisting of:
the position of the elongate member via an elongate member actuator, and
a position of the housing to control the position of the elongate member via the elongate member actuator.

20. (canceled)

21. The method of claim 19, further comprising controlling the tool actuator to control movement of the tool and controlling the elongate member actuator to control movement of the elongate member.

22. The method of claim 17, further comprising:
detecting movement of a distal tip of the elongate member via a sensor; and
controlling the tool actuator to move the tool relative to the elongate member in response to detecting movement of the distal tip of the elongate member.

23. The method of claim 21, further comprising controlling the tool actuator and the elongate member actuator to maintain a position of a distal portion of the tool during a relative movement of the tool and the elongate member that exposes the distal portion of the tool.

24. The method of claim 21, further comprising maintaining a pose of a distal portion of the tool during a relative movement of the tool and the elongate member that exposes a distal portion of the tool.

25. The method of claim 21, wherein the tool is an ablation tool, and further comprising controlling the ablation tool to treat a target tissue after a relative movement of the tool and the elongate member that exposes a distal portion of the ablation tool.

26. The method of claim 15, further comprising translating the tool holder relative to the housing and the elongate member.

27. The method of claim 15, further comprising rotating the tool holder relative to the housing and the elongate member.

28. A medical instrument comprising:
a housing;
an elongate member axially fixed to and extending distally from the housing, the elongate member including a channel extending through the elongate member; and
a tool holder moveably mounted to the housing and configured to accept and retain a tool inserted through the tool holder and into the channel of the elongate member, wherein the tool holder is configured to rotate relative to the housing and the elongate member and rotation of the tool holder rotates the tool relative to the elongate member.
